(19) United States
(12) Patent Application Publication
(10) Pub. No.: US 2003/0088268 A1
(43) Pub. Date: May 8, 2003

(54) Surgical sliding shaft instrument

(76) Inventor: Harald Weinmann, Seitingen-Oberflacht (DE)

Correspondence Address: REIDLAW, L.L.C., 1926 SOUTH VALLEYVIEW LANE, SPOKANE, WA, 99212-0157, US

(21) Appl. No.: 10/322,817

(22) Filed: Dec. 18, 2002

Related U.S. Application Data
(63) Continuation-in-part of application No. 09/654,298, filed on Sep. 1, 2000, now abandoned.

(30) Foreign Application Priority Data
Dec. 17, 1999 (DE) 299 22 271.3

(51) Int. Cl. A61B 17/28

(52) U.S. Cl. 606/205

(57) ABSTRACT

A surgical instrument includes a first grip branch and a second grip branch pivotably connected to the first second grip branch. The second grip branch defines an elongated shaft portion, a grip portion, and a middle piece connecting them. The instrument includes a sliding shaft element which is arranged on the shaft portion and is connectable with the first grip branch. The sliding element is detachable from the shaft portion when the grip branches are at a maximum distance from each other. The instrument has a stopper element to limit travel of the grip branches when in a locking position, and allow maximum splayed movement of the grip branches when in an unlocking position. In one embodiment at least two movement steps in two movement directions are necessary to bring the moveable stopper element from the locking position to the unlocking position.

### CROSS REFERENCE TO RELATED APPLICATIONS

This application is a continuation-in-part of U.S. patent application Ser. No. 09/654,298 filed Sep. 1, 2000, which in turn claims priority under 35 U.S.C. §119 to German Patent application serial number DE 299 22 271.3, filed Dec. 17, 1999.

### FIELD OF THE INVENTION

This invention relates to surgical instruments, and in particular to a scissor-type surgical instrument for gripping objects.

### BACKGROUND

A multitude of surgical instruments exhibit a so called scissor grip which is comprised of two grip branches which can be pivoted towards each other in operation. Examples of such instruments are micro surgical instruments by which the first grip branch is firmly attached to the instrument shaft while the second grip branch is pivotable. Here it can be arranged that the first grip branch must work against a defined spring force while a slide, a punch or a gripper or forceps respectively is or are actuated by pushing together the two grip branches. Such instruments are made de-mountable in order to be able to clean and sterilise them.

It is known from German Patent DE 296 19 246 for this purpose for example that the pivotable bearings of the two grip branches are made to be separable. The two grip branches are attached to each other by means of a bearing pin whereby one grip branch is firmly attached to the bearing pin and the other runs freely on the bearing pin. The disassembly and re-assembly of the scissor grip is achieved in that the bearing pin can be fixed by means of a spring loaded catch device which is arranged radial to its axis. It is therefore possible to disassemble the bearing pin by overcoming the catch effect and pulling the bearing pin out. For this reason a tool or other aid is not needed either for disassembly or for the assembly of the surgical instrument. Equally no particular dexterity or sensitiveness is necessary for inserting and pulling out the bearing pin so that these handholds can be carried out without problems during the clinical routine work.

By disassembling the surgical instrument according to this exposition one obtains at least four separate parts.

It is known from German Patent DE 197 48 396 to join the grip branches with each other in such a manner that they can only be separated from each other by means of a special tool. In addition to this it is intended that the grip branches can be so widely spread apart that a pusher element reaches a position in which the pusher element and the sliding element can be disconnected from each other so that the sliding element can be released from the shaft. Whereby the pusher element is attached to one grip branch and engages with a pusher pin on the sliding element so that by pushing the two grip branches together an axial displacement of the sliding element is achieved. This maximum splayed position can only be reached when a stopper element which is mounted on a grip branch adopts a defined position. During operation the stopper element is located in a locking position in is such a manner that the pusher element can only be located in a defined position in which the pusher pin and pusher element cannot be disconnected from each other. Only by pivoting around an axis of rotation can the maximum splayed grip position of the two grip branches be reached so that then the sliding element can be appropriately removed.

A significant disadvantage of the execution of the pusher element described here is that in the functional position of the surgical instrument the pusher element is only subjected to bending so that here an appropriate static and dynamic dimensioning of the pusher element is necessary.

In addition the pusher element is accommodated in an area of the grip branch in which firstly enough space must be available so that the sliding element can move and also be easily removed. Secondly the pusher element is arranged in an area in which by means of a thumb for example force can be applied to the sliding element. The danger exists that by means of a slight lateral movement of the thumb the stopper element will be pivoted so that the sliding element can unexpectedly loosen itself.

Therefore it is the objective of this invention to provide a device for a surgical instrument without altering the existing design of a known surgical sliding shaft instrument by means of which a simple removal of the sliding element from the shaft is possible.

### SUMMARY

The invention concerns a surgical instrument with a scissor grip, comprised of a first grip branch, a second grip branch, whereby the first grip branch is pivotable or splayable respectively in respect to the second grip branch and can be held at a distance from the other by means of a spring element. The instrument further includes a shaft which is attached to the second grip branch, and a sliding element which is arranged on the shaft and is connectable with the first grip element. The sliding element is detachable from the shaft when in a position in which both grip branches are at a maximum distance from each other and is in the remaining positions axially movable on the shaft by pivoting (pushing together) the first grip element relative to the second grip element. The instrument also has a stopper element to limit the maximum splayed movement of both grip branches, whereby the stopper element is provided with a first position in which the splayed movement is limited and a further position in which the maximum splayed movement is possible, so that in this position the sliding element is detachable from the shaft.

The principle of the invention is to arrange the stopper element in order to limit the splayed movement of the two grip branches in such a manner that even due to improper handling of the surgical instrument an unexpected release of the sliding element is not possible. To achieve this the grip branch which is connected to the pusher element is provided with a stopper element. In order to limit the splayed movement the stopper element contacts the underside of the shaft on which the sliding element is guided.

To achieve the objective of the invention it is recommended that the stopper element be arranged on the first grip branch, that in the first stopper element position the maximum splayed movement be limited by bringing the stopper element in contact with the underside of the shaft and that in a further stopper element position the stopper element can be made to be non-functional in order to attain a maximum splayed movement of the two grip branches.

Here it is of advantage that the stopper element is arranged in a pivotable manner by means of an attachment screw on the first grip branch. The plate-like locking element in its locked position prevents the maximum splayed movement of the two grip elements in that the end of the locking element contacts a part of the underside of the shaft.

To achieve the release of the sliding element and the neutralization of the stopper element function it is preferable that the plate-like stopper element can be moved against a catch-force out of the locking position. Here it is advantageous that a catch element generates the appropriate catch-force. The catch element can be for example a spring loaded pressing piece which engages with a recess on the inside of the locking element.

The arrangement of the locking element on the first grip element has the advantage that firstly a release of the sliding element from the shaft cannot be brought about by improper handling of the surgical instrument and secondly the visual as well as the technical design of the standard model of a surgical sliding shaft instrument remains untouched.

A second embodiment of the invention provides for a surgical instrument for gripping items, which includes a first grip branch and a second grip branch pivotably connected to the first second grip branch. The second grip branch defines an elongated shaft portion, a grip portion, and a middle piece connecting them. The instrument includes a sliding shaft element which is arranged on the shaft portion and is connectable with the first grip branch. The sliding element is detachable from the shaft portion when the grip branches are at a maximum distance from each other. The instrument has a stopper element to limit travel of the grip branches when in a locking position, and allow maximum splayed movement of the grip branches when in an unlocking position. In one embodiment at least two movement steps in two movement directions are necessary to bring the moveable stopper element from the locking position to the unlocking position.

Further advantageous executions can be taken from the following description as well as the drawings.

### DESCRIPTION OF THE DRAWINGS

The drawings show:

FIG. 1 a schematic side view of an execution example of a surgical sliding shaft instrument;

FIG. 2 a partial view of the sliding shaft instrument shown in FIG. 1 in a functional position;

FIG. 3 a partial view of the sliding shaft instrument shown in FIG. 1 in a further functional position;

FIG. 4 a partial view of the sliding shaft instrument shown in FIG. 1 in a disassembled position.

FIG. 5 is a partial side sectional view depicting a sliding shaft instrument in accordance with a second embodiment of the present invention.

FIG. 6 is another partial side sectional view of the sliding shaft instrument depicted in FIG. 5.

FIG. 7 is another partial side sectional view of the sliding shaft instrument depicted in FIGS. 5 and 6.

### DETAILED DESCRIPTION

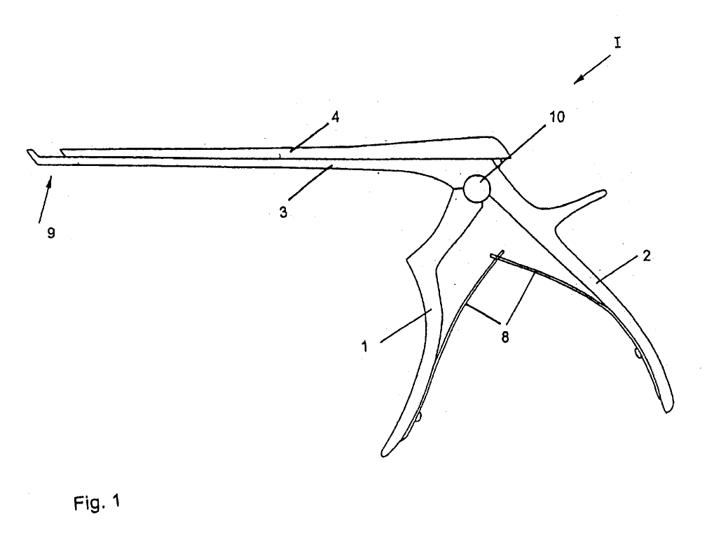

The execution example of a surgical instrument I shown in FIG. 1 is provided with a first grip branch 1 and a second grip branch 2 whereby both grip branches are held at a distance to each other by means of a spring element 8. The two grip branches can be pivoted around a bearing pin 10 towards each other.

In order to push the two grip branches 1,2 together the spring force which is generated by the spring element 8 must be overcome. The shaft 3 is attached to the second grip branch 2 to form one piece. A sliding element 4 is arranged on the shaft 3 in such a manner that it is axially moveable. On the free end of the shaft 3 or the sliding element 4 respectively a punch element 9 for the processing of different materials is provided. The two grip branches 1,2 can be pivoted around a bearing pin 10 towards each other.

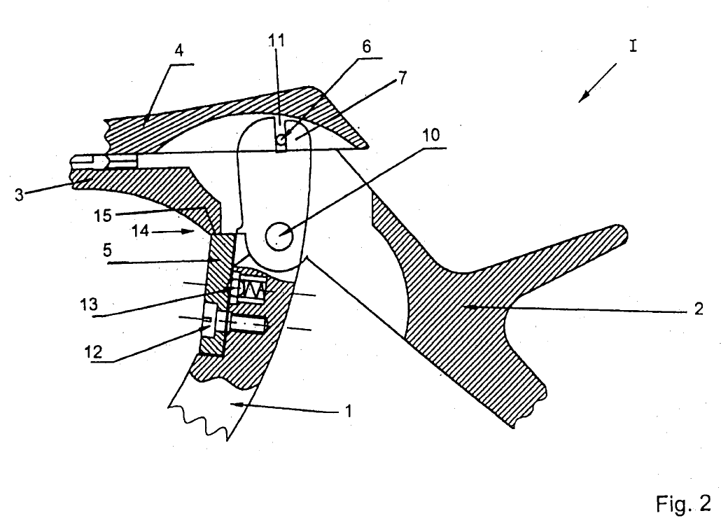

The detail shown in FIG. 2 according to FIG. 1 shows a pusher element 7 which is attached to the first grip branch 1. The pusher element 7 displays a holding device 11 on its free end which in the position shown in FIG. 2 of the two grip branches 1,2 extends into the area of the sliding element 4. The holding device 11 is formed in such a manner that it encompasses a pusher pin 6 which is attached to the sliding element 4 so that it is indirectly connected to the sliding element 4.

In addition a stopper element 5 is arranged by means of an attachment screw 12 on the first grip element 1. In the first grip branch 1 an additional catch element 13 is arranged which holds the stopper element 5 in it's appropriate position. On the end 14 of the stopper element 5 which faces the shaft 3 a stopping face 15 is provided which can be brought into contact with the underside of the shaft 3.

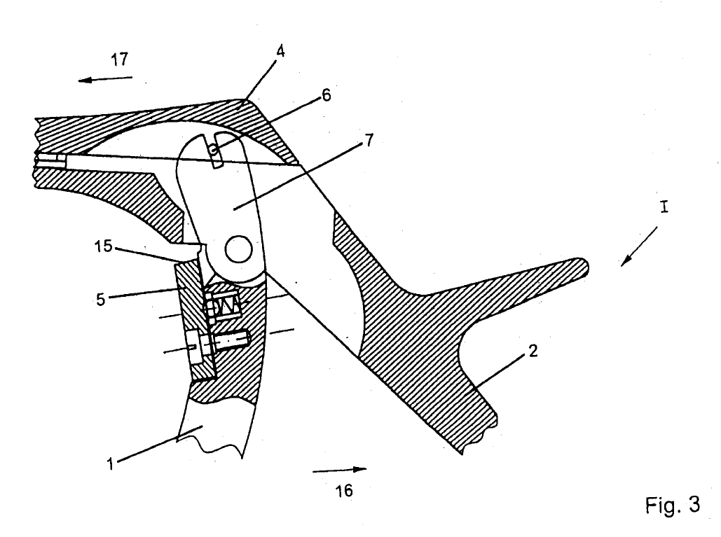

In the position shown in FIG. 3, in which the punch element 9 is open, the stopping face 15 comes into contact with the underside of the shaft 3.

Should the grip branches 1, 2 be pushed together in the direction of the arrow shown in FIG. 3 then the sliding element 4 will be pushed in the direction of arrow 17. This is due to the connection between the first grip branch 1 and the pusher element 7 as well as indirectly the pusher pin 6. Due to this the stopping face 15 of the stopper element 5 is disengaged.

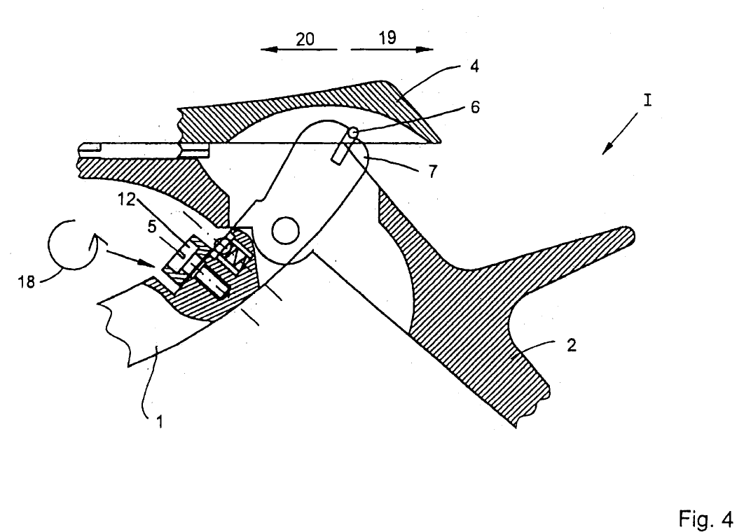

In order to clean the instrument I it is necessary to-remove the sliding element 4 from the rest of the instrument I. For this purpose the stopper element 8 in the position shown in FIG. 3 is to be pivoted in the direction of rotation arrow 18 according to FIG. 4 around the attachment screw 12. In this manner the maximum limited splayed movement as shown in FIG. 2 can be overcome, so that the two grip branches 1, 2 move apart from each other in such a manner that the pusher element 7 is disengaged from the pusher pin 6, so that then the sliding element 4 can be removed in the direction of arrow 19 according to FIG. 4.

To mount the sliding element 4 on the instrument I it is necessary to slip the sliding element 4 onto the shaft 3 in the direction of arrow 20 according to FIG. 4 and this is to be done until the pusher pin 6 connects with pusher element 7.

Next the grip branches 1, 2 are to be pushed together and the stopper element 8 is to be brought again into it's locking position by rotating against the rotation arrow 18 so that the maximum splayed movement of the two grip branches 1, 2 is once again limited.

Due to the arrangement of the stopper element 8 beneath the shaft 3 of grip branch 1 it is possible to develop the instrument in such a way that the original function and the original appearance of a surgical sliding shaft instrument remain unchanged. In addition it has been possible due to the arrangement of the stopper element 8 to prevent that the locking function be unexpectedly cancelled.

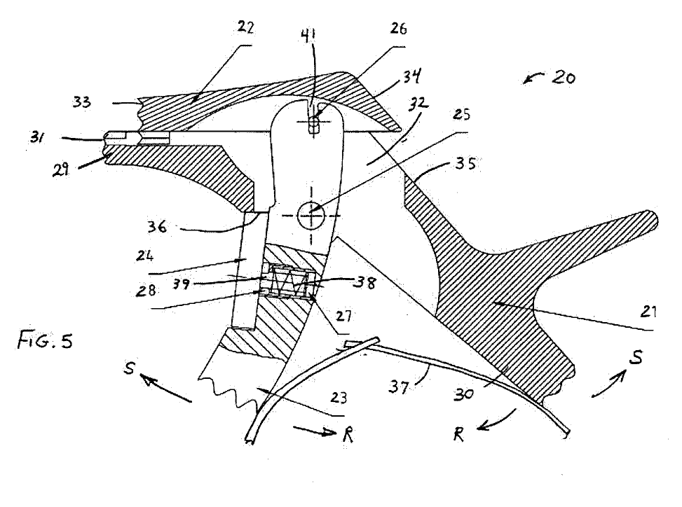

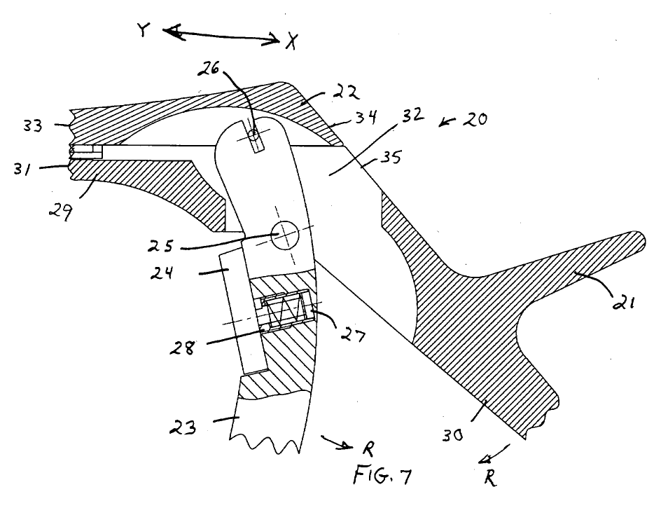

FIGS. 5, 6 and 7 are partial side sectional views depicting a surgical instrument 20 for gripping items in accordance with a second embodiment of the present invention. The instrument 20 is similar to the sliding shaft surgical instrument I depicted in FIGS. 1 through 4 (described above), but includes a different release mechanism. Specifically, with reference now to FIG. 5, the surgical instrument 20 includes a first grip branch 23 and a second grip branch 21, the second grip branch being pivotably connectable to the first grip branch 23 (such as by connector pin 25). The second grip branch 21 defines an extended shaft portion 29, a grip portion 30, and middle piece 32 where the grip portion 30 passes over to the shaft portion 29. The shaft portion 29 of the second grip branch 21 is defined by a shaft free end 31. The instrument 20 further includes a sliding element 22 having a sliding element free 33 end and an opposite end with a front face 34. The sliding element 22 is slidably supported on the shaft portion 29 of the second grip branch 21, and is moveably connectable with the first grip branch 23. In this way, when the first grip branch 23 and the second grip branch 21 are pivotably moved towards one another (in the direction of arrows “R”) from a first position (depicted in FIG. 5) to a second position (depicted in FIG. 7), the sliding element 22 slides on the shaft portion 29 to bring the shaft free end 33 and the sliding element free 31 end in proximity to another. The sliding element 22 can be moveably connected with the first grip branch 23 by a pin 26 supported on the sliding element, which can be received within an open-ended slot defined in an upper end of the first grip branch 23.

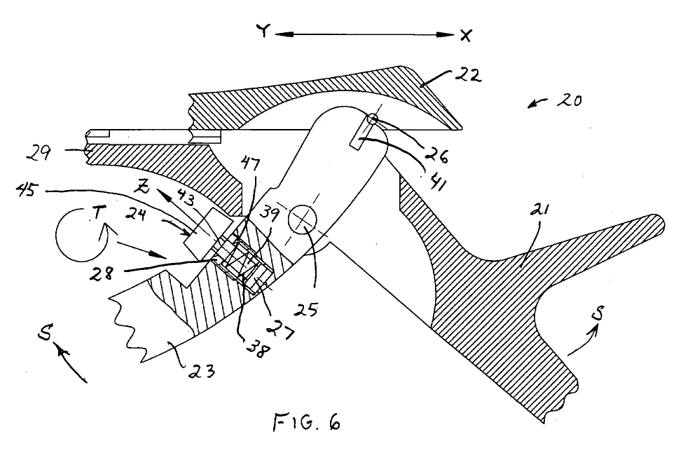

The instrument 20 further includes a moveable stopper element 24 which is supported by one of the first grip branch 23 or the second grip branch 21, and is depicted in FIG. 5 as being supported by the first grip branch 23. The moveable stopper element 24 is moveable from a locking position (depicted in FIG. 5) to an unlocking position (depicted in FIG. 6). In the locking position depicted in FIG. 5, the stopper element 24 prevents the first grip branch 23 and the second grip branch 21 from pivotably moving farther away from one another (in the direction of arrows “S”) than the first position. In the unlocking position depicted in FIG. 6, the stopper element 24 allows the first grip branch 23 and the second grip branch 21 to pivotably move farther away from one another (in the direction of arrows “S”) than the first position (i.e., the position depicted in FIG. 5). When the first grip branch 23 and the second grip branch 21 are pivotably moved away from one another (in directions “S”) from the first position (depicted in FIG. 5) to a second position (depicted in FIG. 6), the sliding element 22 is detachable from the second grip branch 21. Specifically, with respect to the embodiment depicted in FIG. 6, when the first grip branch 23 and the second grip branch 21 are allowed to pivotably move to the second position, the connecting pin 26 on the sliding element 22 can move out of the open end of the slot 41, thus allowing the sliding element 22 to move in direction “X” and off of the second grip branch 21. The instrument 20 can further include a spring element 37 (similar to spring element 8 of FIG. 1) disposed between the first grip branch 23 and the second grip branch 21 to bias the first grip branch and the second grip branch away from another (in the direction of arrows “S” to the first position (depicted in FIG. 5), as well as to a second position (depicted in FIG. 6).

Turning now to FIG. 7, the apparatus 20 is depicted with the first grip branch 21 and the second grip branch 21 being rotatably moved towards one another in direction “Y” until relative movement of the two components is stopped by a gripping force at the tips of the sliding element 22 and the shaft portion 29. The tips of the sliding element 22 and the shaft portion 29 are not shown in FIG. 7, but can resemble the tip 9 of the instrument “I” depicted in FIG. 1. Thus, when the sliding element 22 takes its maximum position in direction “Y” where the shaft free end 31 and sliding element free end 33 are in nearest proximity, the front face 34 of the sliding element and the outer surface 35 of the middle piece 32 of the second grip branch 21 are in perfect alignment. Returning to FIG. 5, it can be seen that when the first grip branch 21 and second grip branch 23 are in the first position (depicted in FIG. 5), then the stopper element 24 is in contact with the underside 36 of the shaft portion 29.

With reference to FIG. 7 (showing the sliding element 22, the first grip branch 23 and the second grip branch in the second of “gripping” position), the front face 34 of the sliding element 22 and the outer surface 35 of the middle piece 32 of the second grip branch 21 form an area where an user can put his or her thumb or finger to register the sliding element 22 with respect to the second grip branch 21. When the grip portion 30 of the second grip branch 21 is defined by a thickness (i.e., the dimension into the sheet of paper on which the figure is drawn), then the area defined by surfaces 34 and 35 can have a width (i.e., the dimension into the sheet of paper on which the figure is drawn) approximately equal to the thickness of the grip portion 30.

With reference to FIG. 5, the stopper element 24 can be configured so that it is only moveable when the first grip branch 23 is nearer to the grip portion 30 of the second grip branch 21 than when in the first position (i.e., when the first grip branch 23 and the grip portion 30 are rotated in directions “R” towards one another from the position depicted in FIG. 5). When the first grip branch 23 and the grip portion 30 are in the “first position” depicted in FIG. 5, the moveable stopper element 24 prevents the first grip branch 23 and the grip portion 30 from moving farther away from one another (in directions “S”) than when in the first position. Further, when the grip branch 23 and the grip portion 30 are in the “first position” depicted in FIG. 5, movement of the stopper element 24 is also blocked.

Preferably, at least two movement steps in two different movement directions are necessary to bring the moveable stopper element 24 from the locking position (depicted in FIG. 5) to the unlocking position (depicted in FIG. 6). Recall that in the locking position (FIG. 5) the stopper element 24 prevents the first grip branch 23 and second grip branch 21 from moving farther away from one another than the “first position” of FIG. 5), whereas when the stopper element 24 is in the unlocking position (FIG. 6) it allows the first grip branch 23 and the second grip branch 21 to moving farther away from one another in directions “S” than in the “first position” of FIG. 5). With respect to FIG. 6, the two movement steps in two different movement directions necessary to bring the stopper element 24 from the locking position to the unlocking position depicted in FIG. 6 can include pulling and turning the stopper element. Specifically, the stopper element can be configured so that it first has to be pulled in direction “Z”, and then rotated in direction “T”, in order to place the stopper element in the position depicted in FIG. 6.

In the embodiment depicted in FIGS. 5 and 6, the stopper element 24 is pivotably supported on the first grip branch 23 by a stem 39 which is attached to knob 45. The stem 39 is received in a recess in the first grip branch. A spring 38 is disposed about the stem 39, and presses against an end member 27 attached to the stem 39, as well as a retaining lip 47 within the recess in the first grip member 23. The spring 38 biases the knob 45 in a direction opposite direction “Z”, so that a positive force is required to allow the knob 45 to be pulled away from the first grip branch 23 in direction “Z”. A key 43 on stem 39 fits in a keyway (formed in keyway member 28) when the stopper element 24 is in the position depicted in FIG. 5. Thus, the key 43 (FIG. 6) prevents rotation of the stopper element 24 in direction “T” when the key 43 is received in the keyway. Thus, to remove the sliding element 22 from the extended shaft portion 29 of the second grip branch 21, the knob 45 of the stopper element 24 must first be pulled in direction “Z” to pull they key 43 out of the keyway member 28, and then the knob must be rotated in direction “T” so that it is moved away from the underside (36, FIG. 5) of the shaft portion 29. This configuration reduces the chance that the stopper element 24 will accidentally be moved to the unlocking position.

In one variation on the embodiment depicted in FIGS. 5 through 7, rather than pulling the knob 45 in direction “Z” to free the key 43 from the keyway 28, the knob 45 can be pressed into a recess in the first grip branch 23 to free the key 43 from the keyway 28. That is, the knob 45 is pressed into the recess in a direction opposite to direction “Z”. In a second variation on the embodiment depicted in FIGS. 5 through 7, rather than pulling the knob 45 in direction “Z” (or pushing it in the opposite direction) to free the key 43 from the keyway 28, the knob 45 can be moved translationally to free the knob for the second rotational movement in direction “T”. For example, the knob 45 can be moved downward (i.e., away from the second extended shaft 29) to free it for the secondary movement in direction “T”. Alternately, the knob 45 can be moved in a direction into or out of the plane of the sheet on which FIG. 6 is drawn in order to free it for secondary movement in direction “T”. In yet another variation, the two movement directions required to place the stopping member 24 in the unlocking position can be translational movement of the knob 45 in a first direction (such as downward away from the shaft 29), followed by translational movement of the knob 45 in a second direction (such as into or out of the plane of the sheet on which FIG. 6 is drawn).

I claim:

1. A surgical instrument for gripping items, comprising:
a first grip branch and a second grip branch, the second grip branch being pivotably connectable to the first grip branch, the second grip branch defining an extended shaft portion, a grip portion, and middle piece where the grip portion passes over to the shaft portion, the extended shaft portion being defined by a shaft free end and an underside, and the middle piece being defined by an outer surface;
a sliding element having a sliding element free end and an opposite end with a front face, the sliding element being slidably supported on the shaft portion and moveably connectable with the first grip branch such that when the first grip branch and the second grip branch are pivotably moved towards one another from a first position to a second position, the sliding element slides on the shaft portion to bring the shaft free end and the sliding element free end in proximity to one another;
a moveable stopper element supported by one of the first grip branch or the second grip branch, the moveable stopper element being moveable from a locking position which prevents the first grip branch and the second grip branch from pivotably moving farther away from one another than the first position, to an unlocking position which allows the first grip branch and the second grip branch to pivotably move farther away from one another than the first position;25 and wherein:
when the first grip branch and the second grip branch are pivotable moved away from one another from the first position to a second position, the sliding element is detachable from the second grip branch; and
when the sliding element takes its maximum position where the shaft free end and sliding element free end are in nearest proximity, the front face of the sliding element and the outer surface of the middle piece of the second grip branch are in perfect alignment and when the first grip branch and second grip branch are in the first position the stopper element is in contact with the underside of the shaft portion.

2. The surgical instrument of claim 1, and wherein the grip portion is defined by a thickness, and further wherein the front face of the sliding element and the outer surface of the middle piece of the second grip branch form an area having a width approximately equal to the thickness of the grip portion, where an user can put its thumb or finger to register the sliding element with respect to the second grip branch.

3. The surgical instrument of claim 1, and wherein the stopper element is only moveable when the first grip branch is nearer to the grip portion of the second grip branch than when in the first position, whereby the moveable stopper element prevents the first grip branch and the grip portion from moving farther away from one another than when in the first position and also blocking movement of the stopper element.

4. The surgical instrument of claim 1, and wherein at least two movement steps in two movement directions are necessary to bring the moveable stopper element from the locking position to the unlocking position.

5. The surgical instrument of claim 3, and wherein the movement steps are pulling and turning.

6. The surgical instrument of claim 3, and wherein the movement steps are pressing and turning.

7. The surgical instrument of claim 3, and wherein the movement steps are translating and turning.

8. The surgical instrument of claim 1, and further comprising a spring element disposed between the first grip branch and the second grip branch to bias the first grip branch and the second grip branch away from another to the first and second positions.

9. The surgical instrument of claim 1, and wherein the stopper element is pivotably supported on the first grip branch and is pivotable from the locking position to the unlocking position.

10. The surgical instrument of claim 7, and wherein:
the first grip branch defines a recess formed therein, the recess further defining a keyway; and
the stopper element comprises:
a knob, and a stem attached to the knob, the stem being slidably receivable within the recess, and fully received within the recess when the stopper element is in the locking position; and
a key defined on the stem and configured to be received within the keyway when the stopper element is in the locking position to thereby resist pivoting of the knob from the locking position to the unlocking position.

11. The surgical instrument of claim 8, and wherein:
the first grip branch includes a retaining lip formed at least partially within the recess; and
the stopper element further comprises an end member and a spring configured to bias the end member away from the retaining lip and thereby bias the knob towards the first grip branch.

12. A surgical instrument for gripping items, comprising:
a first grip branch and a second grip branch, the second grip branch being pivotably connectable to the first grip branch, the second grip branch defining an extended shaft portion, a grip portion, and middle piece where the grip portion passes over to the shaft portion, the extended shaft portion being defined by a shaft free end and an underside, and the middle piece being defined by an outer surface;
a sliding element having a sliding element free end and an opposite end with a front face, the sliding element being slidably supported on the shaft portion and moveably connectable with the first grip branch such that when the first grip branch and the second grip branch are pivotably moved towards one another from a first position to a second position, the sliding element slides on the shaft portion to bring the shaft free end and the sliding element free end in proximity to one another;
a moveable stopper element supported by one of the first grip branch or the second grip branch, the moveable stopper element being moveable from a locking position which prevents the first grip branch and the second grip branch from pivotably moving farther away from one another than the first position, to an unlocking position which allows the first grip branch and the second grip branch to pivotably move farther away from one another than the first position;
when the first grip branch and the second grip branch are pivotable moved away from one another from the first position to a second position, the sliding element is detachable from the second grip branch; and wherein:
at least two movement steps in two movement directions are necessary to bring the moveable stopper element from the locking position to the unlocking position.

13. The instrument of claim 12, and wherein the two movement steps comprise one of:
pulling and turning;
pushing and turning;
translating and turning; or
translating in a first direction and translating in a second direction.
